(19) United States
(12) Patent Application Publication
(10) Pub. No.: US 2020/0399636 A1
(43) Pub. Date: Dec. 24, 2020

(54) Method, system and device for automated NGS library preparation

(71) Applicants: Yan Wang, San Diego, CA (US); Lili Chen, San Diego, CA (US)

(72) Inventors: Yan Wang, San Diego, CA (US); Lili Chen, San Diego, CA (US)

(21) Appl. No.: 16/907,237

(22) Filed: Jun. 20, 2020

Related U.S. Application Data
(60) Provisional application No. 62/863,874, filed on Jun. 20, 2019.

(51) Int. Cl. C12N 15/10; C12Q 1/6806

(52) U.S. Cl. CPC C12N 15/1093; C12Q 1/6806; C12N 15/1013

(57) ABSTRACT

Disclosed is an enclosed magnetic beads-based reaction system for automation of multi-step DNA preparation process involving DNA purification, modification and amplification. It is especially suitable for making DNA library preparation for next-generation sequencing. It uses modules to perform steps of the multi-step reaction and uses magnetic beads to carry DNA to travel through modules to accomplish the multi-step process. The operation can be fully automated, saving time and repetitive hands-on operations and reducing human errors. Using the enclosed reaction system allows parallel processing of different samples and avoids cross-contamination.

### CROSS-REFERENCES AND RELATED APPLICATIONS

This invention claims the benefit of priority to U.S. provisional application No. 62/863,874, filed Jun. 20, 2019, the content of which is hereby incorporated by reference in its entirety.

### FIELD OF THE INVENTION

This invention relates to methods and devices in the field of molecular technologies. It particularly relates to the methods, systems and devices for automated library preparation for next-generation sequencing.

### BACKGROUND OF THE INVENTION

The next-generation sequencing (NGS) is a high throughput sequencing technique that can perform parallel sequencing of a large amount of DNA sequences, which is an important technique used in modern biological research, clinical diagnostics, and forensic sciences. The first step of the next-generation sequencing is to make a DNA library preparation suitable to be used on the NGS platforms. The objective of the NGS library preparation is to add platform-specific sequencing adaptors to the ends of the nucleic acid molecules to be sequenced.

The process of making an NGS library preparation from a DNA/RNA sample involves a series of enzymatic steps, including sample amplification/conversion, end repairing, dA-tailing, adaptor ligation, and product amplification with purification steps in between. The manual operation of making NGS library preparation requires laborious repetitive work and is prone to the risk of cross-contamination and sample loss due to human error. Bulky liquid handling robotics has been used for automation of the NGS library preparation. However, the equipment is expensive and takes large laboratory space. The liquid handling robotics is not cost effective for automation of small numbers of library preparation. Besides, the parallel liquid handling by the robotics cannot avoid cross-contamination effectively.

Therefore, there is a need for development of cost effective, desktop device for handling automated NGS library preparation that can effectively prevent cross-contamination. The present invention satisfies this need and provides other benefits as well.

### SUMMARY OF THE INVENTION

Provided is an enclosed magnetic bead-based reactor system for automation of multi-step DNA preparation process involving purification, modification and amplification. It is especially suitable for making library preparation for next-generation sequencing. It uses modules to perform steps of the multi-step reaction and uses magnetic beads to purify and carry DNA to travel through different modules to accomplish the multi-step process. The operation can be fully automated, saving time and repetitive hands-on operations and reducing human errors. Using the enclosed system allows parallel processing of different samples and effectively avoids cross-contamination.

In an embodiment of the invention, there provides a device for performing automated library preparation for next-generation sequencing, comprising: a) a housing body; b) at least one integrated reactor disposed inside the housing body, wherein the integrated reactor comprises more than one module wherein a module comprises at least one chamber for receiving reaction solutions, samples and magnetic beads, wherein adjacent chambers are separated by a chamber wall, wherein there is an opening at the apex of the chamber wall for allowing magnetic beads to pass through, wherein a chamber can be connected to at least one multi-functional helper (MFH) element; c) a robotic hand for operating the MFH element to move solutions; and d) a magnet for moving the magnetic beads from one chamber to another chamber. The MFH element can have fluid exchange with the connected chamber, wherein the MFH element can function as a solution loader, a mixer and/or a thermally controlled reactor. The MFH element can be connected to the bottom and/or the side of the chamber. The MFH element can be thermally controlled by coupling to a temperature controlling element. The integrated reactor can be sealed with a cover. It can be heat sealed or pressure glued to the cover. The housing body may be equipped with a rotatable seat for holding the integrated reactor so that the integrated reactor can be rotated. The movement of the robotic hand and the magnet, the rotation of the integrated reactor, and the temperature profile of the MFH element can be programmed and automatically controlled by a central controlling element. The housing body can house one or more integrated reactors, each of which can be independently operated through the central controlling element.

In some embodiment, the MFH element uses a springy mechanism to move a solution to or from the connected chamber, and the robotic hand is used to move solutions in and out of the MFH element by exerting a squeezing force.

In some embodiment, the MFH element uses a piston to move a solution to or from the connected chamber, wherein the piston is moved by the robotic hand. In some embodiment, the piston and inner wall of the MFH element forms at least one enclosed section for receiving solutions.

In some embodiment, adjacent modules are connected by a flexible bridge and each module can be manipulated independently. Each module can be independently moved and coupled to its own temperature controlling element.

In some embodiment, the chamber can be a split chamber wherein the split chamber has an internal rib that divides the space inside the chamber into a first and a second compartment for holding two different solutions that can be mixed as needed. When a pellet of magnetic beads is moved into a split chamber, the magnetic beads are first incubated with the solution in the first compartment, and then the split chamber is rotated to mix the solutions and magnetic beads in the first and the second compartments. Optionally, an MFH element can be used to repetitively move the solution in and out of the split chamber to break the pellet of the magnetic beads and ensure thorough mixing of the magnetic beads and the solution.

In some embodiment, the integrated reactor of the present invention comprises at least two modules selected from the following modules in sequential order: a first module, having a single chamber that is connected to at least one MFH element; a second module, having a first chamber, a second chamber and a third chamber, wherein the third chamber of the second module is connected to at least one MFH element; a third module, having a first chamber, a second chamber, and a third chamber, wherein the third chamber is of the third module connected to at least one MFH element; a fourth module, having a first chamber, a second chamber, and a third chamber, wherein the third chamber of the fourth module is connected to at least one MFH element; a fifth module, having a first chamber, a second chamber, and a third chamber, wherein the third chamber of the fifth module is connected to at least one MFH element; and a sixth module, having a first chamber, a second chamber, and a third chamber.

In some embodiment, the integrated reactor comprises optionally the first module, the second module, the third module, the fourth module, optionally the fifth module, and the sixth module.

In some embodiment, the integrated reactor comprises optionally the first module, the third module, the fourth module, optionally the fifth module, and the sixth module.

In some embodiment, the integrated reactor comprises optionally the first module, the fourth module, optionally the fifth module, and the sixth module, wherein the third chamber of the fourth module is connected to two MFH elements or one two-section MFH element.

In some embodiment, the integrated reactor comprises the first module, the fifth module, and the sixth module.

In some embodiment, a module for PCR amplification is inserted after the first module.

In one embodiment of the invention, it provides a method for performing automated library preparation for next-generation sequencing using a library preparation device described herein, comprising: a) loading reaction solutions, magnetic beads and a nucleic acid sample into corresponding chambers or elements; b) sealing the integrated reactor and disposing it into the housing body; c) allowing the nucleic acid sample to react in a first reaction solution to obtain a modified DNA; c) allowing the magnetic beads to bind with the modified DNA; d) sequentially moving the magnetic beads bound with the modified DNA from one chamber to a neighboring chamber and from one module to a neighboring module by use of the magnet, wherein, when the magnetic beads are moved to a chamber with an MFH element, the MFH element is used to add a solution to and/or mix the solution in the connected chamber; and e) collecting the library preparation in the last chamber of the last module.

In some embodiment, each module, if present, in a library preparation device described herein is loaded as follows. The chamber of the first module is loaded with magnetic beads in a bead binding buffer, and the MFH element connected to the first module is loaded with a reaction mix and a nucleic acid sample. The first and the second chamber of the second chamber are loaded with a washing solution, the third chamber of the second module is loaded with an end repairing reaction mix, and the connected MFH element is loaded with a bead binding buffer. The first and the second chamber of the third module are loaded a washing solution, the third chamber of the third module is loaded with a dA-tailing reaction mix, and the connected MFH element is loaded with a bead binding buffer. The first and the second chamber of the fourth module are loaded with a washing solution, the third chamber of the fourth module is loaded with a ligation reaction mix, and the connected MFH element is loaded with a bead binding buffer. The first and the second chamber of the fifth module are loaded with a washing solution, the third chamber of the fifth module is loaded with a polymerase chain reaction (PCR) reaction mix, and the connected MFH is loaded with a bead binding buffer. In some embodiment, the third chamber of the fifth module is connected to a first and a second MFH element. The first element is thermally controlled and left empty, and the second MFH element is loaded with a bead binding buffer. In some embodiment, the third chamber of the fifth module is connected to a thermally controlled MFH element with a first and a second section. The first section is left empty and the second section is loaded with a bead binding buffer. The first and the second chamber of the sixth module are loaded with a washing solution, and the third chamber of the sixth module is loaded with an elution buffer.

In some embodiment, the device is used to make an amplicon library, the reaction mix in the MFH element connected to the first module is a PCR reaction mix, and a PCR amplification is performed in the MFH element connected to the first module to obtain a PCR product, and the PCR product is transferred into the first module.

In some embodiment, the device is used to make a library preparation from a RNA sample, the reaction mix in the MFH element connected to the first module is a reverse transcription retain mix, and a reverse transcription reaction is performed in the MFH element connected to the first module to generate cDNA, and the cDNA is transferred into the first module.

In some embodiment, the third chamber of the fifth module is connected to a first MFH element that is thermally controlled and a second MFH element loaded with a bead binding buffer. The operation in the fifth module comprises the steps of:
a. moving the magnetic beads into the PCR reaction mix in the third chamber of the fifth module;
b. transferring the PCR reaction mix and the magnetic beads to the first MFH element;
c. performing a PCR amplification inside the first MFH element to obtain a PCR product;
d. transferring the PCR product back to the third chamber of the fifth module;
e. using the second MFH element to add a bead binding buffer and mix the magnetic beads, the PCR reaction mix and the bead binding buffer in the third chamber of the fifth module; and
f. using the magnet to pellet the magnetic beads and move them to the next module.

In some embodiment, the third chamber of the fifth module is connected to a thermally controlled MFH element with a first section and a second section. The operation in the fifth module comprises the steps of:
a. moving the magnetic beads into the PCR reaction mix in the third chamber of the fifth module;
b. transferring the PCR reaction mix and the magnetic beads to the first section of the connected MFH element;
c. performing a PCR amplification inside the first section of the connected MFH element to obtain a PCR product;
d. transferring the PCR product, the PCR reaction mix, the magnetic beads in the first section of the connected MFH element, and the bead binding buffer in the second section of the connected MFH element into the third chamber of the fifth module;
e. using the MFH element to mix the solution and the magnetic beads in the third chamber of the fifth module; and
f. using the magnet to pellet the magnetic beads and move them to the next module.

In some embodiment, the library preparation device comprises optionally the first, the second, the third, the fourth, optionally the fifth and the sixth module. When the first module is not present, the library preparation starts from the third chamber of the second module. The third chamber of the second module is loaded with the nucleic acid sample and an end repairing reaction mix, and the connected MFH element is loaded with magnetic beads in a bead binding buffer.

In some embodiment, the starting material is an RNA sample that needs to be converted to single stranded cDNA which needs to be further amplified using a PCR process. In one embodiment, the first module is used for reverse transcription to convert RNA to single stranded cDNA. An additional module, which is used for purification and PCR amplification of the single stranded cDNA, is inserted between the first module and the next module. The added module for PCR amplification can take the same structure as the fifth module, wherein it comprises a first chamber for receiving a washing solution, a second chamber for receiving a washing solution, and a third chamber for receiving a PCR reaction mix, wherein the third chamber is connected to two MFH elements or one two-section MFH element. The third chamber can be connected to a first MFH element that is thermally controlled and left empty, and a second MFH element that is loaded with a bead binding buffer. Or the third chamber can be connected to a thermally controlled two-section MFH element wherein the top section is left empty and the bottom section is loaded with a bead binding buffer. In another embodiment, purification is not needed between the reverse transcription and the PCR amplification, and the two reactions can be sequentially performed in the first module. The first module can be connected to two MFH elements or one two-section MFH element. The chamber of the first module is loaded with a PCR reaction mix, a first MFH element or the top section of the two-section MFH element is loaded with a RNA sample and a reverse transcription reaction mix, and a second MFH element or the bottom section of the two-section MFH element is loaded with a bead binding buffer.

In some embodiment, the library preparation device comprises optionally the first, the third, the fourth, optionally the fifth and the sixth module. When the first module is present, the third chamber of the third module is loaded with an end repairing and dA-tailing reaction mix. When the first module is not present, the library preparation starts from the third chamber of the third module. The third chamber of the third module is loaded with the nucleic acid sample and an end repairing and dA-tailing reaction mix, and the connected MFH element is loaded with magnetic beads in a bead binding buffer.

In some embodiment, the device used for the library preparation comprises the first module, the fourth module, optionally the fifth module, and the sixth module, wherein the third chamber of the fourth module is loaded with an end repairing and dA-tailing reaction mix. In some embodiment, the third chamber of the fourth module is connected to a first and a second MFH element. The first MFH element contains a ligation reaction mix and the second MFH element contains a bead binding buffer. The operation in the fourth module is as follows. The magnetic beads transferred from the first module are washed in the first and the second chamber of the fourth module. They are then incubated with the end repairing and the dA-tailing reaction mix in the third chamber to obtain modified nucleic acids. The ligation reaction mix in the first MFH element is then transferred into the third chamber of the fourth module to react with the modified nucleic acids. After that, the second MFH element is used to add the bead binding buffer to the third chamber, and mix the solution by repetitively moving the mixture of the bead binding buffer, the reaction mix and the magnetic beads in and out of the third chamber so as to break the pellet of the magnetic beads and facilitate DNA to be rebound to the magnetic beads. The magnetic beads are pelleted again and moved to the next module by use of the magnet. In some embodiment, the third chamber of the fourth module is connected to an MFH element having a first and a second section. The first section of the MFH element contains a ligation reaction mix and the second section of the MFH element contains a bead binding buffer. The operation of the fourth module is the same as described above except that the first section and the second section of the connected MFH take the role of the first MFH element and the second MFH element, respectively.

In some embodiment, the device used for the library preparation comprises the fourth module, optionally the fifth module, and the sixth module. Under this situation, the library preparation starts from the third chamber of the fourth module. The third chamber of the fourth module is loaded with an end repairing and dA-tailing reaction mix and a nucleic acid sample. The third chamber of the fourth module is either connected to two MFH elements or one MFH element with two sections. The first MFH element or the first section of the connected MFH element is loaded with a ligation reaction mix. The second MFH element or the second section of the connected MFH element is loaded with magnetic beads in a bead binding buffer. The operation in the fourth module is the same as described above except that the library preparation starts with incubating the nucleic acid sample and the end repairing and dA-tailing reaction mix in the third chamber, and the washing step in the first and second chamber of the fourth module is not needed.

In some embodiment, the device used for the library preparation comprises the first module, the fifth module and the sixth module. The MFH element connected to the first module contains a PCR reaction mix for amplicon generation where target sequences are selectively amplified from the sample. The MFH element connected to the fifth module contains another PCR reaction mix for adding sequencing tags to the DNA products from the first module.

In another embodiment of the invention, it provides a device for performing automated NGS library preparation comprises modules connected by flexible bridges so that each module can be manipulated independently. A chamber inside a module can be a split chamber that contains internal ribs to divide the space inside the chamber into more than one compartment for separately receiving different solutions which can be mixed inside the chamber. The device with flexible bridges and split chambers comprises the following parts: a). a housing body comprising at least one movable unit that can be independently manipulated; b). an integrated reactor comprising at least one module connected by flexible bridges, wherein each module is disposed inside a movable unit and can be independently manipulated by the movable unit, wherein each module comprises at least one chamber for receiving reaction solutions and magnetic beads, wherein the module can be connected to at least one MFH element, wherein at least one chamber is a split chamber; and c). a magnet that can be used to move the magnetic beads from one chamber to another chamber, and from one module to another module via the flexible bridge. The movable unit has a forward tilted position that prevents mixing of different solutions in different compartments of a split chamber and can be rotated to mix the solutions as needed. The rotation of each movable unit and movement of the magnet can be programmed and automatically controlled by a controlling element.

In some embodiment of the device with flexible bridges and split chambers, a module can be thermally coupled to an independent temperature controlling element, allowing the temperature profile of the module to be independently controlled.

In some embodiment of the device with flexible bridges and split chambers, a module can be connected to at least one MFH element, wherein a solution can be transferred between the MFH element and the connected module, and wherein the MFH element can function as a solution loader, a mixer and a thermally controlled reactor.

In some embodiment of the device with flexible bridges and split chambers, the integrated reactor comprises at least two modules selected from the following modules in sequential order: a first module, having a single chamber with one compartment, wherein the module is connected to at least one MFH element; a second module, having a first chamber with one compartment, a second chamber with one compartment and a third chamber with two compartments; a third module, having a first chamber with one compartment, a second chamber with one compartment, and a third chamber with two compartments; a fourth module, having a first chamber with one compartment, a second chamber with one compartment, and a third chamber with two compartments; a fifth module, having a first chamber with one compartment, a second chamber with one compartment, and a third chamber with two compartments, wherein the first compartment of the third chamber of the fifth module is connected to a thermally controlled MFH element at the front side of the chamber; and a sixth module, having a first chamber with one compartment, a second chamber with one compartment, and a third chamber with one compartment. The first, second, third, fourth, fifth and sixth modules are sequentially connected by flexible bridges A, B, C, D and E. The third chamber of the second, the third, the fourth and the fifth module can be optionally connected to a MFH element at the back side of the chamber. The integrated reactor may comprise partial or all of the six modules connected by flexible bridges in the sequential order as specified above. The integrated reactor may comprise, for example, the first module, the second module, the third module, the fourth module, the fifth module, and the sixth module. In another example, the integrated reactor may comprise the first module, the fifth module, and the sixth module.

In one embodiment, the present invention provides a method for performing automated library preparation for next-generation sequencing using a device with flexible bridges and split chambers, comprising: a), loading reaction solutions, magnetic beads and a nucleic acid sample into corresponding chambers or elements; b), sealing the integrated reactor and disposing it into the housing body, wherein all the modules are originally set to a forward tilted position; c), allowing the nucleic acid sample to react in a first reaction solution to obtain a modified DNA; d), allowing the magnetic beads to bind with the modified DNA; e), sequentially moving the magnetic beads bound with the modified DNA from one chamber to a neighboring chamber and from one module to a neighboring module by use of the magnet, and f), collecting the library preparation in the last chamber of the last module. When the magnetic beads are moved into a non-split chamber, the magnetic beads are incubated in the solution of the chamber at the forward tilted position and then moved to the neighboring chamber by use of the magnet. When the magnetic beads are moved into a split chamber, the magnetic beads are incubated with the reaction solution in the first compartment of the split chamber at the forward tilted position. The corresponding module is then rotated back and forth to mix the reaction solution and magnetic beads in the first compartment and the bead binding buffer in the second compartment of the split chamber. Optionally, an MFH element located at the back of the chamber can be used to repetitively move the mixture of the reaction solution, the bead binding buffer and the magnetic beads in and out of the chamber so as to break the pellet of the magnetic beads and facilitate DNA molecules to be rebound to the magnetic beads. The DNA bound magnetic beads are pelleted again and moved to the neighboring module via the connecting flexible bridge. The magnetic beads are moved through each module in a similar manner until reaching the last module where the library preparation is eluted from the magnetic beads.

In some embodiment, each module, if present in the device with flexible bridges and split chambers, is loaded as the following: the chamber of the first module is loaded with magnetic beads in a bead binding buffer, and the MFH element connected to the first module is loaded with a reaction mix and the nucleic acid sample; the first and the second chamber of the second chamber are loaded with a washing solution, the first compartment of the third chamber of the second module is loaded with an end repairing reaction mix, and the second compartment of the third chamber of the second module is loaded with a bead binding buffer; the first and the second chamber of the third module are loaded with a washing solution, the first compartment of the third chamber of the third module is loaded with a dA-tailing reaction mix, and the second compartment of the third chamber of the third module is loaded with a bead binding buffer; the first and the second chamber of the fourth module are loaded with a washing solution, the first compartment of the third chamber of the fourth module is loaded with a ligation reaction mix, and the second compartment of the third chamber of the fourth module is loaded with a bead binding buffer; the first and the second chamber of the fifth module are loaded with a washing solution, the first compartment of the third chamber of the fifth module is loaded with a PCR reaction mix, and the second compartment of the third chamber of the fifth module is loaded with a bead binding buffer; and the first and the second chamber of the sixth module are loaded with a washing solution, and the third chamber of the sixth module is loaded with an elution buffer.

In some embodiment, the operation in the fifth module with a split chamber is as follows: after being washed in the first and the second chamber of the fifth module, the magnetic beads are mixed with the PCR reaction mix in the first compartment of the third chamber of the fifth module, the magnetic beads and the PCR reaction mix are then transferred to the thermally controlled MFH element that is connected to the fifth module. A PCR amplification is performed inside the connected MFH element to obtain a PCR product, and the PCR product and the magnetic beads are transferred back to the first compartment of the third chamber of the fifth module. The fifth module is then rotated back and forth to mix the reaction solutions and magnetic beads in the first and the second compartment. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. The magnetic beads are pelleted again and moved to the sixth module via the flexible bridge E.

In some embodiment, the device with flexible bridges and split chambers comprises optionally the first module, the second module, the third module, the fourth module, optionally the fifth module, and the sixth module. When the first module is not included in the device, the library preparation starts from the third chamber of the second module. The first compartment of the third chamber of the second module is loaded with the nucleic acid sample and an end repairing reaction mix, and the second compartment of the third chamber of the second module is loaded with magnetic beads in a bead binding buffer.

The starting material may be an RNA sample that needs to be converted to single stranded cDNA which needs to be further converted to double stranded DNA and be amplified using a PCR process. In some embodiment, the first module is used for reverse transcription to convert RNAs to single stranded cDNAs. An additional module is inserted after the first module, which is used for PCR amplification of the single stranded cDNA. The chamber of the first module is loaded with magnetic beads in a bead binding buffer, and it is connected to an MFH element loaded with an RNA sample and a reverse transcription mix. The added module for PCR amplification comprises a first chamber for receiving a washing solution, a second chamber for receiving a washing solution, and a third split chamber wherein the first compartment of the third split chamber is loaded with a PCR reaction mix and the second compartment of the third split chamber is loaded with a bead binding buffer, wherein a thermally controlled MFH element is connected to the first compartment of the third chamber of the added module. In some embodiment, purification is not needed between the reverse transcription and the PCR amplification, and the two reactions can be sequentially performed in the first module, wherein the chamber of the first module is a split chamber. The first compartment of the split chamber is loaded with a PCR reaction mix, and it is connected with an MFH element loaded with an RNA sample and a reverse transcription mix. The second compartment of the split chamber is loaded with magnetic beads in a bead binding buffer.

In some embodiment, the device with flexible bridges and split chambers comprises the first module, the third module, the fourth module, optionally the fifth module, and the six module, wherein the first compartment of the third chamber of the third module is loaded with an end repairing and dA-tailing reaction mix, and the second compartment of the third chamber of the third module is loaded with a bead binding buffer.

In some embodiment, the device with flexible bridges and split chambers comprises the third module, the fourth module, optionally the fifth module, and the six module. Under this situation, the library preparation starts from the third chamber of the third module. The first compartment of the third chamber of the third module is loaded with the nucleic acid sample, an end repairing and dA-tailing reaction mix, and the second compartment of the third chamber of the third module is loaded with magnetic beads in a bead binding buffer.

In some embodiment, the device with flexible bridges and split chambers comprises the first module, the fourth module, optionally the fifth module, and the sixth module, wherein the first compartment of the third chamber of the fourth module is loaded with an end repairing and dA-tailing reaction mix, and the second compartment of the third chamber of the fourth module is loaded with a bead binding buffer. An MFH element containing a ligation reaction mix is connected to the first compartment of the third chamber of the fourth module at the front side, and an MFH element acting as a mixer can be optionally connected to the third chamber of the fourth module at the back side of the chamber. The operation in the fourth module is as follows. After being washed in the first and the second chamber of the fourth module, the magnetic beads are incubated with the end repairing and dA-tailing reaction mix in the first compartment of the third chamber of the fourth module to obtain modified nucleic acids. The ligation reaction mix in the connected MFH element is then transferred into the first compartment of the third chamber of the fourth module to react with the modified nucleic acids. The fourth module is rotated back and forth to mix the solutions and the magnetic beads in the first and second compartment of the third chamber of the fourth module. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. The magnetic beads are pelleted again and moved to the fifth module via the flexible bridge D.

In some embodiment, the device with flexible bridges and split chambers comprises the fourth module, optionally the fifth module, and the sixth module. Under this situation, the library preparation starts from the third chamber of the fourth module. The first compartment of the third chamber of the fourth module is loaded with an end repairing and dA-tailing reaction mix and a nucleic acid sample, and the second compartment of the third chamber of the fourth module is loaded with magnetic beads in a bead binding buffer. Additionally, an MFH element containing a ligation reaction mix is connected to the first compartment of the third chamber of the fourth module at the front side, and an MFH element acting as a mixer can be optionally connected to the third chamber of the fourth module at the back side of the chamber. The operation in the fourth module is as follows: the nucleic acid sample is first incubated with the end repairing and dA-tailing reaction mix in the first compartment of the third chamber of the fourth module to obtain modified nucleic acids. The ligation reaction mix in the MFH element is then transferred into the first compartment of the third chamber of the fourth module to react with the modified nucleic acids. The fourth module is rotated back and forth to mix the reaction solution and in the first compartment and the magnetic beads and the bead binding buffer in second compartment of the third chamber of the fourth module. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. The magnetic beads are pelleted and moved to the fifth module via the flexible bridge D.

In some embodiment, the device with flexible bridges and split chambers comprises the first module, the fifth module and the sixth module. The MFH element connected to the first module contains a PCR reaction mix for amplicon generation where target sequences are selectively amplified from the sample using target specific primers. The MFH element connected to the fifth module contains another PCR reaction mix for adding sequencing tags to the DNA products from the first module.

### BRIEF DESCRIPTION OF DRAWINGS

FIG. 1A shows an embodiment of a six-module reactor without flexible bridges; and FIG. 1B shows an embodiment of a six-module reactor with flexible bridges.

FIG. 2A shows an embodiment of an MFH element with an elastic bag in a closed mode; and FIG. 2B shows an embodiment of an MFH element with an elastic bag in an open mode.

FIGS. 3A-3C shows an embodiment of a one-section MFH element with a piston in solution loading mode (FIG. 3A), a close mode (FIG. 3B) and solution releasing mode (FIG. 3C).

FIGS. 4A-4E shows an embodiment of a two-section MFH element with a piston in a mode for loading solution to the bottom section (FIG. 4A), a mode for loading solution to the top section (FIG. 4B), a close mode (FIG. 4C), a mode for releasing solution in the top section, (FIG. 4D) and a mode for releasing solution in the bottom section (FIG. 4E).

FIG. 5 shows an example of DNA library preparation protocol using a six-module device.

FIG. 6 shows an example of DNA library preparation protocol using a five-module device.

FIG. 7 shows an example of DNA library preparation protocol using a four-module device.

### DETAILED DESCRIPTION

Definitions: Unless otherwise defined, all technical and scientific terms used herein have the same meaning as commonly understood by one of the ordinary skills in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The term “integrated reactor”, as used herein, refers to a multi-unit container for holding different reaction reagents in different units so that a series of reactions can be sequentially carried out in these units. Each unit of the integrated reactor is called a “module”, which is composed of at least one “chamber”, usually multiple chambers, for holding reaction solutions to carry out a series of related steps in a functional step. For example, the library preparation comprises functional steps of end repairing, dA-tailing, and adaptor ligation which can be carried out in an end repairing module, a dA-tailing module, and an adaptor ligation module, respectively. The end repairing step comprises, for example, a first wash, a second wash, and an end-repairing enzymatic reaction. The end repairing module comprises a first chamber, a second chamber and a third chamber for carrying out the first wash, the second wash, and the end-repairing enzymatic reaction, respectively. A module can be added or removed from the device depending on the protocol of interest. The function of multiple modules can also be combined into a single module as needed. In one embodiment, modules are positioned next to each other. In other embodiment, neighboring modules are separated by a flexible bridge so that each module can be independently manipulated. Chambers are separated from each other by chamber walls, wherein each chamber has an enclosed section so that no direct fluid exchange can occur between two chambers. There is an opening on the apex of the chamber wall which allows magnetic beads to pass from one chamber to its neighboring chamber. A non-split chamber is a type of chamber that encloses an undivided “compartment” wherein only one solution can be held in the chamber. A split chamber has a internal rib that divides the space inside the chamber into two partially separate compartments wherein different solutions can be held in the two compartments. The internal rib does not completely block the liquid communication between the two compartments within a split chamber. The solutions in the two compartments of a split chamber can be temporarily held separate and mixed together as needed.

The term “multi-functional helper (MFH) element”, as used herein, refers to a multi-functional accessory part that can be connected to the chambers of the device which provides a mechanism to move liquid solution to and from the connected chamber. The MFH element can function as a solution loader that transfers a solution in the MFH element to the connected module. The MFH element can also function as a mixer by repetitively moving a solution in and out of the connected module. Additionally, the MFH element can be thermally coupled to a temperature controlling element so that it can be used as a thermally controlled reactor. A nucleic acid sample and a reaction solution can be pre-loaded in an MFH element, and a thermal reaction (e.g. a PCR amplification or a reverse transcription) can be performed in the MFH element. The product of the thermal reaction can then be transferred to the connected module. Alternatively, the solution in the connected chamber can be transferred to the MFH element to perform a thermal reaction, and the product of the thermal reaction can then be transferred back to the connected chamber. The liquid exchange between the chamber and the MFH element can be controlled by an open/close mechanism, for example, using a clamp or a valve. The MFH element shall provide a simple mechanism for moving liquids in and out of the connected chamber. For example, the MFH element may comprise a piston that is used to push liquids to and draw liquids from the connected chamber. The MFH element may comprise an elastic bag which can push liquid out when an external squeezing force is exerted, and provide a vacuum to attract liquid in when the external force is released.

Disclosed is an enclosed magnetic bead-based system/device for performing multi-step reactions, suitable for processes such as extraction, purification and modification of nucleic acid and protein samples, wherein target molecules affixed to magnetic microparticles are sequentially moved through a series of reaction solutions to undergo different reactions. The system is especially useful for making DNA library preparation for next-generation sequencing. The magnetic bead-based system comprises an integrated reactor with multiple modules, which is used to carry out a series of functional steps of library preparation, including sample amplification/conversion, end-repairing, dA-tailing, adaptor ligation, and product amplification. A module comprises of at least one chamber, usually multiple chambers, for holding reaction solutions to carry out a series of related steps in a functional step. A chamber can be connected to one or more MFH elements, which function as a solution loader, a mixer and/or a thermally controlled reactor. Using the MFH element enables loading additional solution to a chamber and mixing the solution in the chamber, which allows multiple reactions to be carried out sequentially in the same chamber. This feature is important since it allows for off-bead enzymatic modification of target DNAs and the rebound of the target DNAs to magnetic beads in the same chamber, which is essential for the DNA library preparation process. DNA molecules are unbound from magnetic beads in low salt buffers (e.g. end repairing reaction mix, dA-tailing reaction mix, and ligation reaction mix), and DNA molecules need to be rebound to magnetic beads before they can be carried by the magnetic beads to the next chamber. After an enzymatic reaction, an MFH element can be used to add a bead binding buffer and mix the bead binding buffer, the enzymatic reaction buffer and magnetic beads to ensure that DNA molecules are effectively rebound to magnetic beads. Additionally, the MFH element can be thermally coupled to a temperature controlling element and used as a thermally controlled reactor for performing reactions such as PCR and reverse transcription. Using MFH elements for performing thermal reactions is more flexible and saves more energy compared to making the whole integrated reactor to be thermally controlled.

The present invention provides a device for performing automated library preparation for next-generation sequencing, comprising: a). a housing body; b). at least one integrated reactor disposed inside the housing body, which comprises at least one module wherein a module comprises at least one chamber for receiving solutions and magnetic beads, wherein adjacent chambers are separated by a chamber wall, wherein there is an opening at the apex of the chamber wall for allowing magnetic beads to pass through, wherein a chamber can be connected to at least one MFH element; c). a robotic hand for operating the MFH element; and d). a magnet for moving the magnetic beads from one chamber to another chamber. The integrated reactor can be sealed with a cover. It can be heat sealed or pressure glued to the cover. The MFH element can be thermally coupled to a temperature controlling element. The housing body may be equipped with a rotatable seat for holding the integrated reactor so that the integrated reactor can be rotated. The movement of the robotic hand and the magnet, the rotation of the integrated reactor, and the temperature profile of the MFH element can be programmed and automatically controlled by a central controlling element. The housing body can house one or more integrated reactors, each of which can be independently operated through the central controlling element. Each integrated reactor can be used for different library preparations without causing cross-contamination among different samples.

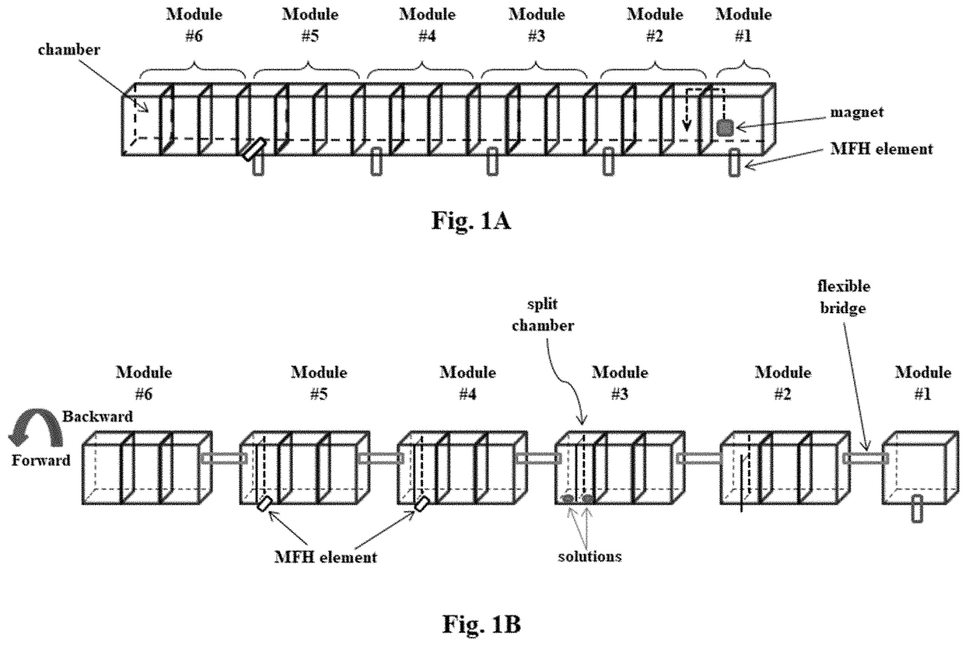

An integrated reactor comprises multiple modules which comprises at least one chamber, usually multiple chambers, for holding reaction solutions to carry out a series of related reactions in a functional step. A module can be placed next to the adjacent module (FIG. 1A) or be separated from its neighbor by a flexible bridge (FIG. 1B). Depending on the protocol of choice, a module can be added, combined or removed from the integrated reactor to be adapted to the need of a particular protocol. For example, a typical DNA library preparation protocol can comprise up to six functional steps carried out in different modules of the integrated reactor, including sample amplification/conversion (module #1), end repairing (module #2), dA-tailing (module #3), adaptor ligation (module #4), PCR amplification of ligated product (module #5) and final elution (module #6). Except for the first module, all the other modules have more than one chamber. A chamber has an enclosed section for receiving reaction solutions, magnetic beads and samples. Adjacent chambers within a module are separated by a chamber wall so that solutions in adjacent chambers cannot be mixed. There is a small opening on the top of the chamber wall, wherein magnetic beads can pass through the top opening of the chamber wall and be transferred from one chamber to its adjacent chamber. The shape of the chamber is not limited, including, for example, a cube, a cubiod and a cylinder. The edges and corners of the chamber can be rounded up to avoid potential dead corners for trapping magnetic beads. Although the device is exemplified with its application in DNA library preparation, the target molecules to be used with the device are not limited to nucleic acids, but include a variety of substances such as proteins, peptides, ligands and other molecules that can be affixed to magnetic beads. The magnetic beads can be coated with a material to facilitate the binding to target molecules. For example, the magnetic beads can be coated with protein A or protein G for binding to antibodies. The magnetic beads can be coated with an antibody for isolating the antibody-specific antigens. Streptavidin-coated magnetic beads can be used to separate biotin-labeled molecules from the rest of the substances. Once the target molecules are affixed to magnetic beads, they can be used in the device of the invention to undergo a series of reactions for modifications and purification as described herein.

A significant aspect of the invention is to use an MFH to function as a solution loader, a mixer and/or a thermally controlled reactor. An MFH is a multi-functional accessory part that can have controlled fluid exchange with a connected chamber. An MFH element can function as a solution loader that transfers a solution in the MFH element to the connected chamber. An MFH element can also function as a mixer by repetitively moving a solution in and out of the connected chamber. Additionally, a MFH element can be thermally coupled to a temperature controlling element so that it can be used as a thermally controlled reactor. The MFH element incorporates a close and an open mechanism that allows the fluid exchange between the MFH element and the connected chamber to be controlled. The open/close mechanism can be, for example, a clamp or a valve that can be open and closed. The MFH element can be thermally coupled to a temperature controlling element, which can be, for example, a thin film electrode heater, a metal block heater or a Peltier device that can provide heating or cooling as needed. This allows the MFH element to function as a thermally controlled reactor where a PCR amplification, a reverse transcription reaction, or other thermal reactions can be performed.

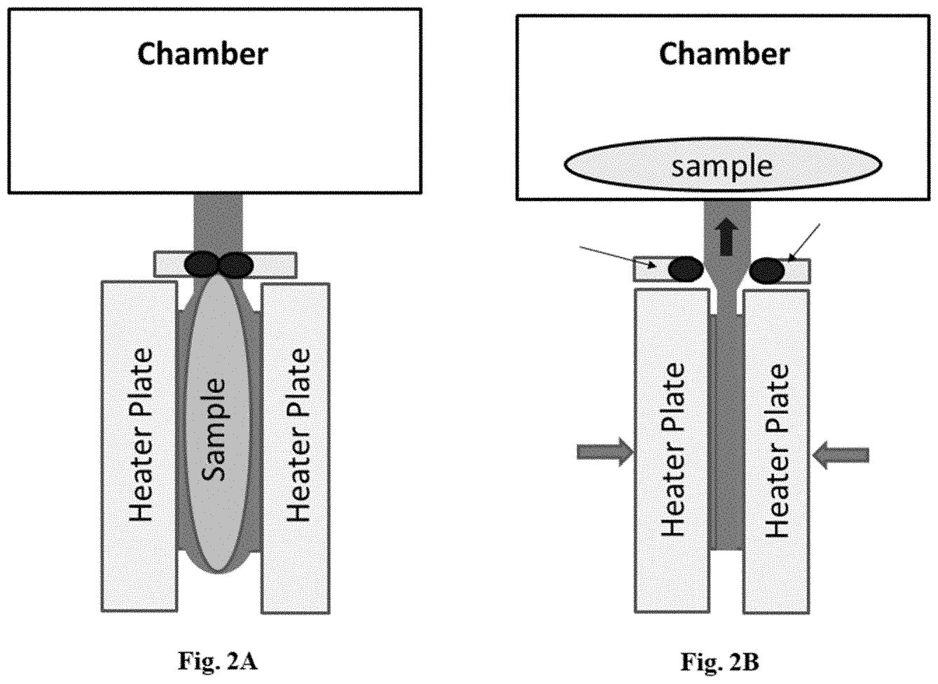

The MFH element provides a simple mechanism for moving solutions to and from the connected module. In one embodiment, the MFH element uses a springy mechanism to transfer solutions to and from the connected chamber (FIGS. 2A & 2B). The MFH element comprises an elastic container (e,g, an elastic bag) that has an inherent tendency to open when there is no external force and a clamp for controlling the fluid exchange. During the close mode (FIG. 2A), the clamp is closed and there is no fluid exchange between the MFH element and the connected chamber. During the open mode (FIG. 2B), the clamp is open and fluid exchange between the MFH element and the connected chamber is enabled. When the elastic container is compressed by an external force, the solution inside the inner container can be squeezed out of the MFH element and into the connected chamber. When the elastic container is released from the external force, the inner container springs open and generate a vacuum space to withdraw solution from the connected chamber. The external force can be operated by a robotic hand that can be programmed to move to different modules to exert forces to any MFH element of interest. The robotic hand can press on two outside plates to apply an external force to squeeze the solution out of the inner elastic container and into the connected chamber. When the external force is released, the elastic container is open, creating a vacuum force to draw the solution back to the MFH element. By repetitively applying and releasing the external force, the solution can be repetitively moved in and out the chamber to achieve the mixing function. The outside plate can also be heating blocks that can be used to control the temperature profile of the inner elastic container.

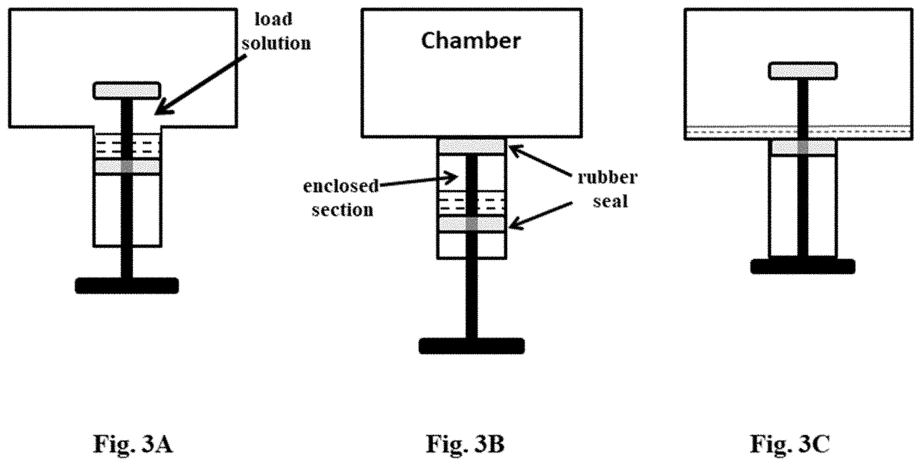

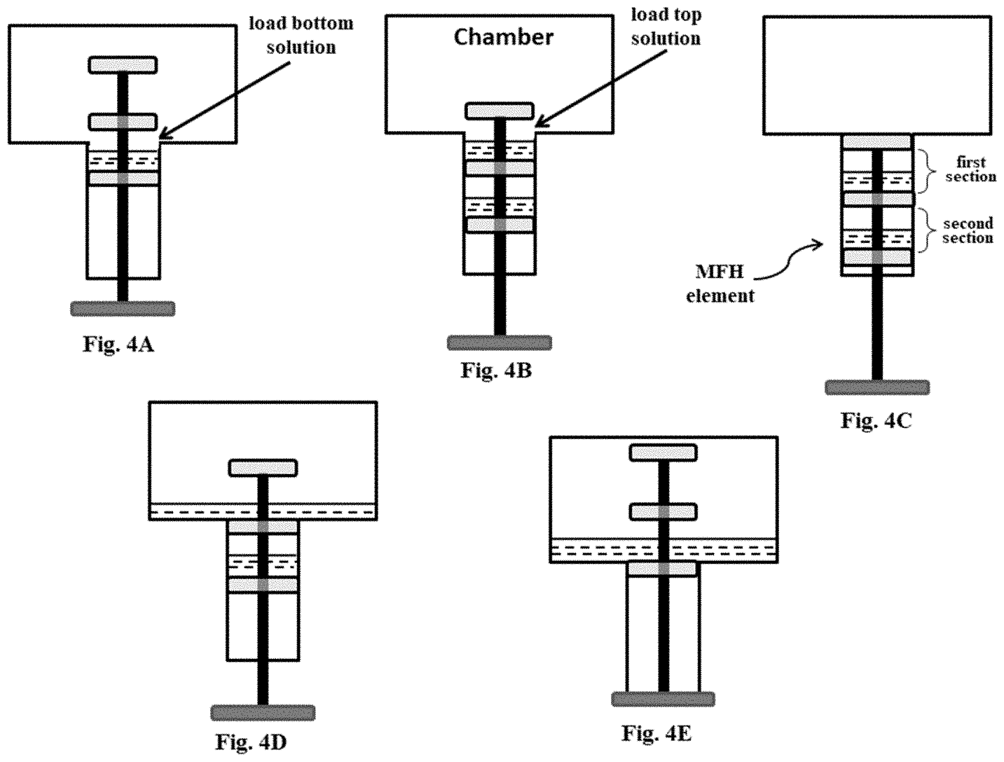

In some embodiment, the present invention provides an MFH element comprising a barrel and a piston with multiple rubber seals (FIGS. 3A-3C and 4A-4E), wherein the rubber seals and the inner wall of the barrel can form one or more enclosed sections for receiving solutions. The solution in the enclosed section of the MFH element can be moved into the chamber by pushing the piston to an open position. The solution in the chamber can be pulled into the enclosed section by pulling the piston to a close position. The rubber seal and the inner wall of the barrel forms a water-proof seal so that no water leakage between different sections will occur when the MFH element is at the close mode. FIGS. 3A-3E show a one-section MFH element which uses a piston with two rubber seals to form one enclosed section for receiving one solution. FIGS. 4A-4E shows a two-section MFH element that uses a piston with three rubber seals to form two enclosed sections for receiving two different solutions. When the piston is pushed to a loading position, a solution can be loaded into the section formed by the rubber seal and the inner wall of the barrel (FIGS. 3A, 4A and 4B). When the piston is pulled to a close position, the MFH element is closed to the chamber and the solution is maintained inside the enclosed section of the MFH element (FIGS. 3B and 4C). The piston can be pushed to an open position to release the solution in the enclosed section of the MFH element (FIG. 3C). The piston can also be pushed to a first open position to release the top solution only (FIG. 4D) and to a second open position to release the bottom solution (FIG. 4E). The piston can be repetitively moved between the closed position and the first open position for selectively mixing the solution in the top section of the MFH element with the solution in the connected chamber. The piston can be repetitively moved between the close position and the second open position for mixing the solution in the bottom section of the MFH element with the solution in the connected chamber. The movement of the piston can be controlled by the robotic hand which is controlled by the central controlling element. The two-section MFH element enables sequentially adding two different solutions into the connected chamber. For example, the first solution can be a ligation reaction mix and the second solution can be a bead binding buffer.

A chamber can be connected to more than one MFH element. The MFH element can be connected to the bottom and/or the side of the chamber. The magnet and the MFH element are preferably positioned at the different side of a chamber. For example, the magnet can be positioned at the back side of a chamber, and the MFH element can be connected to the front side of a chamber. When performing a liquid exchange between a chamber and the MFH element on the side, the integrated reactor can be rotated so that the MFH element is at a favorable position for liquid exchange. For example, the integrated reactor can be forward rotated for 90° so that the side MFH element is facing downward, and a robotic hand positioned underneath the chamber can be used to exert external force to control the liquid exchange between the MFH element and the connected chamber.

To operate the magnetic bead-based system, magnetic beads, reaction solutions, and samples are pre-loaded into the appropriate chambers or MFH elements of the integrated reactor, and the reactor is sealed by a sealing element. To start the reaction series, the sample can be directly bound to the magnetic beads or react with a first reaction solution before being bound to the magnetic beads. Once the magnetic beads are bound with target molecules, a magnet is used to pellet and move the target-bound magnetic beads from one chamber to the next chamber, and from one module to the next module. Sometimes target-bound magnetic beads are moved into a reaction solution that renders target molecules to be unbound from magnetic beads. After the off-bead reaction is finished, an MFH element is used to add a bead binding buffer to the chamber. The same MFH element is further used to thoroughly mix the bead binding buffer, the reaction mix and the magnetic beads to facilitate the rebound of target molecules to the magnetic beads. After the target molecules are rebound to the magnetic beads, the target-bound magnetic beads are pelleted again and moved to the adjacent chamber. By this way, the target-bound magnetic beads are sequentially moved from one chamber to the next chamber and from one module to the next module, thereby carrying out a series of reactions to obtain the final product. The operation of the device can be fully automated, saving time and repetitive hands-on operations and reducing human errors. Using the enclosed system allows parallel processing of different samples and avoids cross-contamination.

The process of making a NGS library preparation from a DNA/RNA sample often involves a series of enzymatic steps, including sample amplification/conversion, end repairing, dA-tailing, adaptor ligation, and product amplification with purification steps in between. The end repairing process uses a DNA polymerase to produce blunt ended dsDNAs and a DNA kinase to phosphorylate their 5′ ends. The dA-tailing process uses a DNA polymerase to add a single dAMP residue to the 3′ end of the blunt ended dsDNAs. The adaptor ligation process uses a DNA ligase to ligate a sequencing adaptor to the dsDNA fragments with a 3′ AMP overhang. Depending on a variety of factors, the library preparation process can be performed in many alternative ways. For example, the end repairing and dA-tailing process can be combined into one step. The end repairing, dA-tailing and adaptor ligation can be performed sequentially without purification. The library preparation can be performed with or without PCR amplification. Additionally, the starting material may come from different sources that requires different handling. For example, the source materials could be fragmented genomic DNA, selected DNA sequences, cDNA sequences, total RNA, and selected RNA sequences. The library preparation device should be adaptable to the requirements of all these different protocols.

The magnetic bead-based NGS library preparation device comprises multiple functional modules, allowing to choose different combinations of modules to satisfy the requirements of different library preparation protocols. In some embodiment, the NGS library preparation device comprises at least an integrated reactor, comprising modules selected from the following six modules: a first module having a single chamber, wherein the chamber of the first module is connected to at least one MFH element; a second module having a first, a second and a third chamber, wherein the third chamber of the second module is connected to an MFH element; a third module having a first, a second and a third chamber, wherein the third chamber of the third module is connected to an MFH element; a fourth module having a first, a second and a third chamber, wherein the third chamber of the fourth module is connected to at least an MFH element; a fifth module having a first, a second and a third chamber, wherein the third chamber of the fifth module is connected to two MFH elements or a two-section MFH element; and a sixth module, having a first, a second and a third chamber.

The library preparation protocol proceeds in an ascending numerical order from the first module to the second module until to the sixth module, and from first chamber to the second chamber to the third chamber within a module. If one module is not selected, the rest of the modules are connected in the same ascending numerical order with the same numeric notation. For example, if the first module is missing, the integrated reactor comprises the second module, the third module, the fourth module, the fifth module and the sixth module, and the library preparation protocol proceeds from the second module to the third module until to the six modules. If the second and fifth modules are missing, the integrated reactor comprises the first module, the third module, the fourth module, and the sixth module, and the library preparation protocol proceeds from the first module to the third module to the fourth module to the sixth module.

The first module is used for PCR amplification of a DNA sample or reverse transcription reaction of a RNA sample. The first module comprises one chamber for receiving magnetic beads in a bead binding buffer, and at least one MFH element which is loaded with a DNA/RNA sample and a reaction mix for performing a PCR amplification or a reverse transcription reaction. The magnetic beads used in DNA library preparation are referred to magnetic microparticles that can non-specifically bind and unbind to DNA/RNA under certain salt concentrations and polyethylene glycol (PEG) concentrations. The salt can be sodium chloride, magnesium chloride, calcium chloride, potassium chloride, lithium chloride, barium chloride or cesium chloride. By adjusting the salt concentration and the PEG concentration, one can render DNA molecules to bind to free magnetic microparticles or elute DNA molecules from the DNA bound magnetic microparticles. Although magnetic microparticles has a preferred shape of beads, the shape of the magnetic microparticles is not limited, which can be regular or irregular shape. This type of magnetic beads is well known in the art, for example, magnetic microparticles disclosed in U.S. Pat. No. 5,705,628, and are commercially available from many vendors (e.g. AMPure beads from Beckman Coulter (Brea, Calif.) and MagJet magnetic beads from Thermo Fisher Scientific (Waltham, Mass.)). The magnetic beads can effectively bind to DNA molecules in solutions with high salt concentrations (e.g. 0.5M-5M) and 7-30% PEG. The bead binding buffer comprises higher concentrations of salt and PEG (e.g. 5M sodium chloride and 20% PEG) so that when it is mixed with other reaction solution, the mixed solution has salt and PEG concentrations that enables DNA to bind to the magnetic beads.

When the starting material is DNA, for example, genomic DNA and circulating cell-free DNA, a PCR amplification can be performed to selectively amplify DNA molecules of interest (e.g. amplicon amplification). When the starting material is RNA, for example, total RNA and mRNA, a reverse transcription reaction is performed to convert RNA to cDNA, which can be further amplified by a PCR amplification. The method for converting RNA to cDNA and using PCR amplification to make double stranded DNA is well known in the art (Head S R, Komori H K, LaMere S A, Whisenant T, Van Nieuwerburgh F, Salomon D R, Ordoukhanian P. Biotechniques 2014, 56(2):61-4). The PCR amplification or the reverse transcription reaction of a sample can be performed before loading into the first module, or be carried out in the MFH elements connected to the first module. Once a DNA product is made via a reverse transcription reaction and/or a PCR amplification in the MFH element, the DNA product and the reaction mix is transferred to the first module, mixed with the bead binding buffer and the magnetic beads to allow the DNA product to be bound with the magnetic beads. After the DNA product is bound with the magnetic beads, the magnet is used to pellet the magnetic beads and move the pellet into the first chamber of the next module.

In some embodiment, the starting material is an RNA sample that needs to be converted to single stranded cDNA which needs to be further amplified using a PCR process. For this situation, the first module is used for reverse transcription to convert RNAs to single stranded cDNAs. An additional module can be inserted between the first module and the next module, which is used for PCR amplification of the single stranded cDNA. The chamber of the first module is loaded with magnetic beads in a bead binding buffer and it is connected to an MFH element loaded with a RNA sample and a reverse transcription mix. The added module for PCR amplification comprises a first chamber for receiving a washing solution, a second chamber for receiving a washing solution, and a third chamber for receiving a PCR reaction mix, wherein the third chamber is connected to one MFH element with two-sections or two MFH elements. In some embodiment, the third chamber of the added module is connected to two MFH elements with a first MFH element that is thermally controlled and left empty, and a second MFH element that is loaded with a bead binding buffer. In some embodiment, the third chamber of the added module is connected to a thermally controlled MFH element with two enclosed sections, wherein the top section is left empty and the bottom section is loaded with a bead binding buffer. The RNA sample is first converted to single stranded cDNA and bound to magnetic beads in the first module as described above. The DNA bound magnetic beads are then moved to the added module for PCR amplification, wherein the cDNA is purified and amplified by PCR in a similar way as that of the fifth module. If purification is not needed between the reverse transcription and the PCR amplification, the two reactions can be sequentially performed in the first module. In one embodiment, the first module is connected with two MFH elements. The chamber of the first module is loaded with the PCR reaction mix, a first connected MFH element is loaded with the RNA sample and the reverse transcription mix, and a second connected MFH is loaded with magnetic beads in a bead binding buffer. The RNA sample is first converted to the single stranded cDNA in the first MFH using a reverse transcription reaction. The reverse transcription mix and the cDNA product is then mixed with the PCR reaction mix in the chamber of the first module. The mixture is then transferred back to the first MFH element where the PCR amplification is performed. After the PCR amplification, the solution in the first MFH element is transferred to the chamber of the first module, where it is further mixed with the magnetic beads and the bead binding buffer in the second MFH element. After that, a magnet is used to pellet the magnetic beads and move them to the next module. In another embodiment, a two-section MFH element is used in lieu of two MFH elements. The first and the second section of the two-section MFH element performs the same function of the above first and the second MFH element, respectively.

The second module is used for the end repairing process to ensure that the DNA molecules to be sequenced have a blunted end with a 5′ phosphate end and a 3′ hydroxyl end. The second module comprises a first chamber for receiving a washing solution, a second chamber for receiving a washing solution, and a third chamber for receiving an end repairing reaction mix. The MFH element connected to the third chamber of the second module contains a bead binding buffer. The MFH element is preferably positioned underneath the chamber. The washing solution, for example, 60-80% ethanol, is a solution used to wash away impurities, such as proteins, polysaccharides and lipids, that are bound to magnetic beads. The end repairing reaction mix comprises a DNA polymerase to produce blunt ended dsDNAs and a DNA kinase to phosphorylate their 5′ ends in an appropriate buffer that can maintain the activity of both the DNA polymerase and the DNA kinase. The DNA polymerase suitable for this purpose has 5′->3′ DNA polymerase activity, optionally 3′->5′ exonuclease activity but no 5′->3′ exonuclease activity, including, for example, T4 DNA polymerase and Klenow large fragment of E. coli DNA polymerase I. The DNA kinase suitable for this purpose has the activity of incorporating a phosphate to the 5′end of dsDNA, including, for example, T4 DNA kinase. The buffer suitable for maintaining the activity of the DNA polymerase and DNA kinase are well known in the art. The end repairing reaction mix is commercially available in the NGS library preparation kit such as SPARK™ sample DNA prep kit (Qiagen, Hilden, Germany).

When the DNA-bound magnetic beads are transferred into the first chamber of the second module, the magnetic beads are washed in the washing solution to remove impurities bound to the beads. The magnetic beads are then moved into the washing solution in the second chamber for a second wash, after which the magnetic beads are moved to the end repairing reaction mix in the third chamber. The DNA molecules are unbound from the magnetic beads in the end repairing reaction mix as it has low salt concentration without PEG. The DNA molecules are incubated the end repairing reaction mix at room temperature (e.g. 10-30 minutes) to produce blunt ended dsDNAs and add a phosphate group to the 5′ end of the dsDNA. After the end repairing process is finished, the connected MFH element is open and the bead binding buffer is transferred into the third chamber of the second module. The MFH element is further used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads and move them into the first chamber of the next module.

In some NGS library preparation protocol, a PCR amplification or a reverse transcription reaction is not needed (e.g. the starting material is fragmented dsDNA) and the first module is not needed. Under this situation, the library preparation protocol starts from the third chamber of the second module. The first and the second chamber of the second module may be present and be left empty, or the first and the second chamber may be removed from the second module. The dsDNA sample is directly added with the end repairing reaction mix in the third chamber of the second module, and magnetic beads in a bead binding buffer are loaded in the MFH element connected to the third chamber of the second module. The dsDNA sample is first reacted with end repairing enzymes to obtain modified DNAs in the third chamber of the second module. After the end repairing process, the rest of operation is the same as described above.

The third module is used for a dA-tailing process which uses a DNA polymerase to add a single dAMP residue to the 3′ end of the blunt ended dsDNAs. The third module comprises a first chamber for receiving a washing solution, a second chamber for receiving a washing solution, and a third chamber for receiving a dA-tailing reaction mix. The MFH element connected to the third chamber is loaded with a bead binding buffer. The dA-tailing reaction mix comprises a DNA polymerase, for example, Taq DNA polymerase or Klenow fragment without exonuclease activity, that can incorporate a dAMP residue to the 3′ end of a blunt ended dsDNA. The dA-tailing process produces dsDNA molecules with a single 3′ dAMP overhang that can facilitate efficient ligation of sequencing adaptors to the ends of the dsDNAs.

When the DNA-bound magnetic beads are transferred into the first chamber of the third module, the magnetic beads are washed in the washing solution to remove impurities. The magnetic beads are then moved into the washing solution in the second chamber for a second wash, after which the magnetic beads are moved to the dA-tailing reaction mix in the third chamber. The DNA molecules are unbound from the magnetic beads in the dA-tailing reaction mix as it has low salt concentration without PEG. The DNA molecules are incubated in the dA-tailing reaction mix at room temperature (e.g. 10-30 minutes) to produce dsDNAs with a single dAMP overhang at the 3′ end. After the dA-tailing process is finished, the MFH element is used to transfer the bead binding buffer into the third chamber. The MFH element is further used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads and move them into the first chamber of the next module.

In some embodiment, the end repairing process and the dA-tailing process can be combined together in one module and the second module is not needed. Under this situation, when the first module is present, the first module is directly connected to the third module. The end repairing enzymes and the dA-tailing enzymes added together in the same buffer makes the end repairing and dA-tailing reaction mix, which is loaded into the third chamber of the third module. The operation can be performed in analogy to the operations described above. When the first module is not present, the library preparation protocol starts with the third chamber of the third module. The nucleic acid sample, the end repairing enzymes and dA-tailing reaction mix are loaded together in the third chamber of the third module. Magnetic beads in a bead binding buffer are loaded into the MFH element connected to the third chamber of the third module. The operation can be performed in analogy as described above.

The fourth module is used to ligate platform specific sequencing adaptors to the ends of dsDNA with 3′ overhangs. The fourth module comprises a first chamber for receiving a washing solution, a second chamber for receiving a washing solution, and a third chamber for receiving a ligation reaction mix. The MFH element connected to the third chamber is loaded with a bead binding buffer. The ligation reaction mix comprises a DNA ligase, for example, T4 DNA ligase and E. coli DNA ligase, that can facilitate the joining of DNA strands together by catalyzing the formation of a phosphodiester bond.

When the DNA-bound magnetic beads are transferred into the first chamber of the fourth module, the magnetic beads are washed in the washing solution to remove impurities. The magnetic beads are then moved into the washing solution in the second chamber for a second wash, after which the magnetic beads are moved to the ligation reaction mix in the third chamber. The DNA molecules are unbound from the magnetic beads in the ligation reaction mix as it has low salt concentration without PEG. The DNA molecules are incubated in the ligation reaction mix at room temperature (e.g. 10-60 minutes) to produce dsDNAs with sequencing adaptors. After the ligation process is finished, the MFH element is used to transfer the bead binding buffer into the third chamber. The MFH element is further used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads and move them into the first chamber of the next module.

In some embodiment, the end repairing, the dA-tailing and DNA ligation process can be combined in one module, and the second and the third module is not needed. If the first module is present, it is positioned next to the fourth module. The third chamber of the fourth module is loaded with the end repairing and dA-tailing reaction mix. In some embodiment, the third chamber of the fourth module is connected to two MFH elements, with a first MFH element loaded with a ligation reaction mix and a second MFH element loaded with a bead binding buffer. After the DNA-bound magnetic beads are washed, the magnetic beads are incubated with the end repairing and dA-tailing reaction mix to complete the end repairing and dA-tailing process together. After that, the ligation reaction mix in the first MFH element is transferred into the third chamber to perform the ligation reaction. After the ligation reaction is completed, the second MFH element is used to transfer the bead binding buffer into the third chamber. It is further used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads and move them into the first chamber of the next module.

In some embodiment, the third chamber of the fourth module is connected to an MFH element with two sections, wherein the top section is loaded with a ligation reaction mix and the bottom section is loaded with a bead binding buffer. The operation in the fourth module is the same as described above except that the top section and the bottom section of the MFH element take the role of the above first and the second MFH element, respectively.

If the first module is not present, the library preparation protocol starts from the third chamber of the fourth module. Under this situation, the DNA sample, the end repairing reaction and dA-tailing reaction mix is loaded into the third chamber of the fourth module. The ligation reaction mix is loaded into the first MFH element or the top section of the connected MFH element. The magnetic beads in the bead binding buffer are loaded into the second MFH element or the bottom section of the connected MFH element. The operation in the fourth module can be performed in analogy as described above.

The fifth module is used for PCR amplification of the dsDNA molecules with sequencing adaptors obtained from the processes above. This module is optional in the library preparation protocols. It is used to increase the amount of the output DNA if the amount of starting material is low. It can also be used to add barcodes and other oligos to target DNAs to be sequenced. In order to minimize the PCR amplification bias, the cycle number of the PCR amplification should be set to be low, for example, it can be set to be 4-6. When the fifth module is not present, the fourth module is directly connected to the six modules. The fifth module comprises a first chamber for receiving a washing solution, a second chamber for receiving a washing solution, and a third chamber for receiving a PCR reaction mix. The PCR reaction mix comprises a DNA polymerase, a mixture of four dNTPs, a pair of primers designed to be complementary to the adaptor sequences that are ligated to the target DNA molecules. In some embodiment, the third chamber of the fifth module is connected to two MFH elements with a first MFH element that is thermally controlled and left empty, and a second MFH element that is loaded with a bead binding buffer. When the DNA-bound magnetic beads are transferred into the first chamber of the fifth module, the magnetic beads are washed in the washing solution to remove impurities. The magnetic beads are then moved to the washing solution in the second chamber for a second wash, after which the magnetic beads are moved into the PCR reaction mix in the third chamber. The PCR reaction mix and the magnetic beads are then transferred into the first connected MFH element to perform the PCR amplification. After the PCR amplification, the DNA product and the PCR reaction mix are transferred back to the third chamber of the fifth module. The second MFH element is used to transfer the bead binding buffer into the third chamber. The MFH element is further used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads and move them into the first chamber of the sixth module. In some embodiment, the third chamber of the fifth module is connected to a thermally controlled MFH element with two enclosed sections, wherein the top section is left empty and the bottom section is loaded with a bead binding buffer. The operation in the fifth module is the same as described above except that the top section and the bottom section of the MFH element take the role of the above first and the second MFH element, respectively.

The sixth module is used to elute clean dsDNA molecules with sequencing adaptors from the magnetic beads. The six module comprises a first chamber for receiving a washing solution, a second chamber for receiving a washing solution, and a third chamber for receiving an elution buffer. The elution buffer is a solution/liquid used to release pure DNA molecules from the magnetic beads. It can be pure water or TE buffer containing 10 mM Tris-HCl, pH 7-9. When the DNA-bound magnetic beads are transferred to the first chamber of the sixth module, the magnetic beads are washed in the washing solution to remove impurities. The magnetic beads are then moved into the washing solution in the second chamber for a second wash. The DNA-bound magnetic beads are then moved into and incubated in the elution buffer in the third chamber, wherein the final DNA product is released from the magnetic beads and dissolved in the elution buffer. The magnetic beads are then pelleted and separated from the elution buffer. The final DNA library preparation is obtained from the elution buffer.

In some embodiment, the library preparation protocol uses two PCR amplifications to add sequencing tags to target DNA. For this protocol, the library preparation device comprises the first module, the fifth module and the sixth module. The first module is used to perform a PCR amplification to selectively amplify target sequences of interest. The target sequence-bound magnetic beads are directly moved to the fifth module to perform a second PCR amplification that adds sequencing adaptors to the target sequences. Finally, the target sequences tagged with sequencing adaptors are eluted in the sixth module.

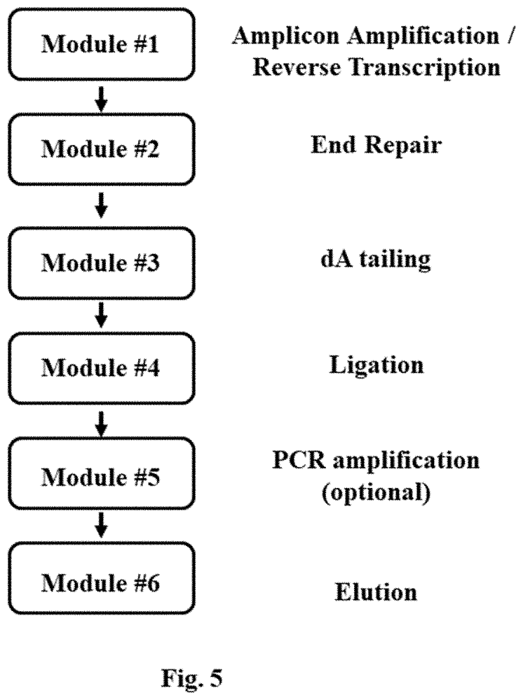

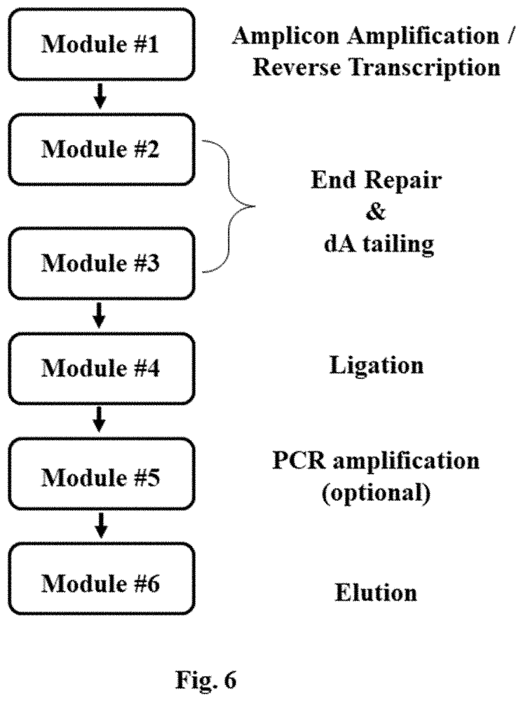

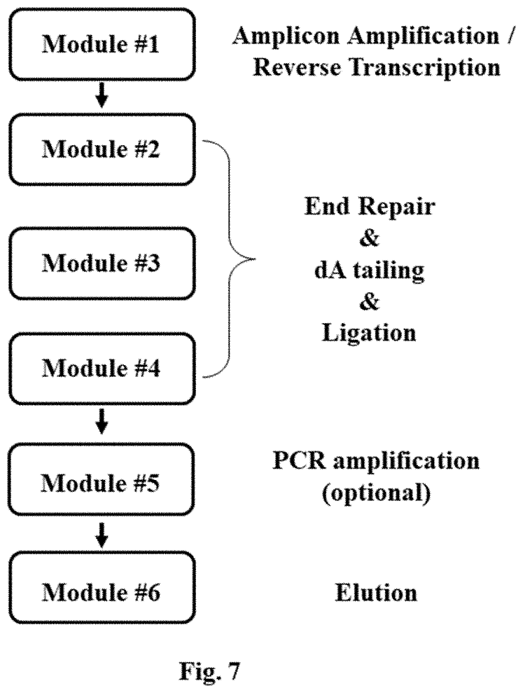

The NGS library preparation device has a modular structure, making it flexible and adaptable to satisfy the requirements of different library preparation protocols. For a standard library preparation protocol comprising separate end repairing, dA-tailing and adaptor ligation steps, the device may comprise the second, the third and the fourth module. For protocols requiring an initial PCR amplification or reverse transcription, the first module can be included in the device. The fifth module is an optional module used for amplification of the ligated DNA product. For example, FIG. 5 shows a protocol comprising a first module for PCR amplification or reverse transcription of a sample, a second module for end repairing process, a third module for dA-tailing process, a fourth module for adaptor ligation process, an optional fifth module for PCR amplification of DNA product, and a sixth module for elution of the DNA library preparation. In some NGS library preparation protocol, the end repairing and the dA-tailing process can be combined in one module, that is, the second and the third module are combined into one module. To simplify the naming system, the combined module is called the third module while the second module is removed. FIG. 6 shows a protocol comprising a first module for PCR amplification or reverse transcription of a sample, a third module for the end repairing and dA-tailing process, a fourth module for the adaptor ligation process, an optional fifth module for PCR amplification of DNA product, and a sixth module for elution of the DNA library preparation. In some NGS library preparation protocol, the end repairing, the dA-tailing and adaptor ligation process can all be combined in one module. The combined the module is named the fourth module and the second and third modules are removed. FIG. 7 shows a protocol comprising a first module for PCR amplification or reverse transcription of a sample, a fourth module for end repairing, dA-tailing and adaptor ligation process, an optional fifth module for PCR amplification of DNA product, and a sixth module for elution of the DNA library preparation.

In another embodiment, the present invention provides a magnetic bead-based system for NGS library preparation, comprising an integrated reactor having more than one independently operated modules connected by flexible bridges. Each module can be independently rotated or thermally controlled without affecting the other modules. This modular design allows separating the multi-step reaction into groups of functional units, each of which can be independently controlled. A module comprises one or more chambers for receiving fluids used in different reaction steps. One type of chamber, called non-split chamber, encompasses only one single undivided compartment that allows holding only one solution. Another type of chamber, called split chamber, has an internal rib that divides the space inside the chamber into two partially separate compartments that allows separately holding two different solutions, which can be mixed together as needed. The split chamber provides a mechanism for sequential performance of two reactions in the same chamber. This allows for the target molecules to be unbound and rebound to the magnetic beads in the same chamber, an essential process for DNA library preparation. A module can be also connected to one or more MFH elements, which can function as a solution loader, a mixer and a thermally controlled reactor. This adds flexibility and diversity to the reaction system.

The NGS library preparation device comprises a housing body which contains more than one movable units that can be independently manipulated, an integrated reactor with more than one modules connected by flexible bridges, wherein each module is disposed into a movable unit of the housing body, and a magnet that can move along the outside wall of the integrated reactor to move magnetic beads inside the integrated reactor. The movable unit with a module disposed inside can be rotated back and forward to mix solutions inside the module. Because the modules are connected by flexible bridges, the movement of one module can be carried out without affecting the neighboring modules. The flexible bridges are made of materials that allows easy bending and twisting without breaking the structure. Using a flexible bridge to connect two modules allows each module to be independently rotated by the attached movable unit without affecting the position of the neighboring module. The flexible bridge shall have a smooth surface to facilitate the movement of magnetic beads. It can have a length of 10-40 mm wherein the length of the flexible bridge shall be sufficient so that the movement of two neighboring modules are without spatial hindrance.

Each module has at least one chamber with an enclosed section for receiving reaction solutions, magnetic beads, and nucleic acid samples. Chambers within a module is separated by a chamber wall so that solutions in adjacent chambers cannot be mixed. There is a small opening on the top of the chamber wall, wherein magnetic beads can pass through the top opening of the chamber wall and be transferred from one chamber to the adjacent chamber. A non-split chamber has an undivided space for receiving one solution only. A split chamber has an internal rib that divides the space inside the chamber into two partially separated compartments for receiving two different solutions that can be mixed together when needed. The split chamber is originally set to be forward tilted position so that different solutions in the two compartments are not mixed. When mixing of the solutions is needed, the module is rotated back and forth by the movable unit until the solutions in the two compartments are mixed.

A chamber of a module can be connected to MFH elements that are multi-functional accessory parts. An MFH element can function as a solution loader that transfers a solution in the MFH element to the connected module. An MFH element can also function as a mixer by repetitively moving a solution in and out of the connected module. Additionally, a MFH element can be thermally coupled to a temperature controlling element so that it can be used as a thermally controlled reactor.

To operate the magnetic bead-based NGS library preparation system, magnetic beads, reaction solutions, and samples are pre-loaded into the appropriate compartments or MFH elements of the integrated reactor, and the integrated reactor is sealed by a sealing element. To start the reaction series, the sample can be directly bound to the magnetic beads or react with a first reaction solution before being bound to the magnetic beads. Once the magnetic beads are bound with target molecules, a magnet is used to pellet and move the target-bound magnetic beads from one chamber to the next chamber, and from one module to the next module. When the magnetic beads are moved into a non-split chamber, the magnetic beads are incubated in the solution of the chamber at the tilted position and then moved to a neighboring chamber by use of the magnet. When the magnetic beads are moved into a split chamber, the magnetic beads are incubated with the buffer in the first compartment of the split chamber at the forward tilted position, and the corresponding module is then rotated back and forth to mix the solution and magnetic beads in the first and the second compartment of the split chamber. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. The magnetic beads are pelleted again and moved to the neighboring module via the connecting flexible bridge. In the split chamber, target molecules are allowed to be unbound from the magnetic beads in the first compartment, and be rebound to the magnetic beads when a bead binding buffer in the second compartment is mixed in. Using the MFH element as a mixer can help break the pellet of the magnetic beads to ensure complete mixing of the DNA, the magnetic beads, and the bead binding buffer. This step facilitates the rebound of DNA to the magnetic beads to ensure a good DNA recovery. The target-bound magnetic beads are sequentially moved from one chamber to the next chamber and from one module to the next module, thereby carrying out a series of reactions to obtain the final product.

The process of making a NGS library preparation from a DNA/RNA sample involves a series of enzymatic steps and the library preparation process can be performed in a variety of ways. The NGS library preparation device comprises multiple independently manipulated modules, allowing to choose different combinations of modules to satisfy the requirements of different library preparation protocols. In some embodiment, the NGS library preparation device can select from the following six modules: a first module having a single chamber with one compartment, wherein the module is connected to at least one MFH element, a second module having a first chamber with one compartment, a second chamber with one compartment and a third chamber with two compartments, a third module having a first chamber with one compartment, a second chamber with one compartment, and a third chamber with two compartments, a fourth module having a first chamber with one compartment, a second chamber with one compartment, and a third chamber with two compartments, a fifth module having a first chamber with one compartment, a second chamber with one compartment, and a third chamber with two compartments, wherein the third chamber of the fifth module is connected to a thermally controlled MFH element, and a sixth module, having a first chamber with one compartment, a second chamber with one compartment, and a third chamber with one compartment. The first, second, third, fourth, fifth and sixth modules are sequentially connected by flexible bridges A, B, C, D and E. The third chamber of the second, the third, the fourth, and the fifth module can be optionally connected to an MFH element at the back side of the chamber.

The first module is used for PCR amplification of a DNA sample or reverse transcription reaction of a RNA sample. The first module comprises a non-split chamber for receiving magnetic beads in a bead binding buffer, and at least one MFH element which is loaded with a DNA/RNA sample and a reaction mix for performing a PCR amplification or a reverse transcription reaction. Once the PCR amplification or reverse transcription is completed in the connected MFH element, the DNA product and the reaction mix is transferred to the first module, mixed with the bead binding buffer and the magnetic beads to allow the DNA product to be bound with the magnetic beads. After the DNA product is bound with the magnetic beads, the magnet is used to pellet the magnetic beads to the bottom of the first module that is close to the flexible bridge A, and guide the magnetic beads pellet to travel through the flexible bridge A into the first chamber of the next module.

The starting material may be an RNA sample that needs to be converted to single stranded cDNA which needs to be further converted to double stranded DNA and be amplified using a PCR process. In some embodiment, the first module is used for reverse transcription to convert RNAs to single stranded cDNAs. An additional module is inserted after the first module, which is used for PCR amplification of the single stranded cDNA. The chamber of the first module is loaded with magnetic beads in a bead binding buffer, and it is connected to an MFH element loaded with an RNA sample and a reverse transcription mix. The added module for PCR amplification comprises a first chamber for receiving a washing solution, a second chamber for receiving a washing solution, and a third split chamber wherein the first compartment of the third split chamber is loaded with a PCR reaction mix and the second compartment of the third split chamber is loaded with a bead binding buffer, wherein a thermally controlled MFH element is connected to the first compartment of the third chamber of the added module. The RNA sample is first converted to single stranded cDNA and bound to magnetic beads in the first module as described above. The DNA bound magnetic beads are then moved to the first and the second chamber of the added module for washing away the impurities. The magnetic beads are then moved to the PCR reaction mix in the first compartment of the third chamber. The magnetic beads and the PCR reaction mix are transferred into the connected MFH element to perform a PCR amplification. After the PCR amplification is completed, the reaction mix and the magnetic beads are transferred back to the first compartment of the third chamber and mixed with the bead binding buffer in the second compartment. After that, the magnetic beads are pelleted and moved to the next module.

In some embodiment, purification is not needed between the reverse transcription and the PCR amplification, and the two reactions can be sequentially performed in the first module, wherein the chamber of the first module is a split chamber. The first compartment of the split chamber is loaded with a PCR reaction mix, and it is connected with an MFH element loaded with a RNA sample and a reverse transcription mix. The second compartment of the split chamber is loaded with magnetic beads in a bead binding buffer. The RNA sample is first converted to the single stranded cDNA in the connected MFH element using a reverse transcription reaction. The cDNA product and the reverse transcription mix is then transferred to the first compartment of the split chamber where they are mixed with the PCR reaction mix. The mixture is then transferred back to the connected MFH element where the PCR amplification is performed. After the PCR amplification, the solution in the connected MFH element is transferred to the first compartment, and it is further mixed with the magnetic beads and the bead binding buffer in the second compartment. After that, a magnet is used to pellet the magnetic beads and move them to the next module.

The second module is used for the end repairing process to ensure that the DNA molecules to be sequenced have a blunted end with a 5′ phosphate end and a 3′ hydroxyl end. The second module comprises a first chamber for receiving a washing solution, a second chamber for receiving a washing solution, and a third split chamber having a first compartment for receiving an end repairing reaction mix and a second compartment for receiving a bead binding buffer.

When the DNA-bound magnetic beads are transferred into the first chamber of the second module, the magnetic beads are washed in the washing solution to remove impurities bound to the beads. The magnetic beads are then moved into the washing solution in the second chamber for a second wash, after which the magnetic beads are moved to the end repairing reaction mix in the first compartment of the third chamber. The DNA molecules are unbound from the magnetic beads in the end repairing reaction mix as it has low salt concentration without PEG. The DNA molecules are incubated with the end repairing reaction mix at room temperature to produce blunt ended dsDNAs and add a phosphate group to the 5′ end of the dsDNA. All these operations are performed when the second module is arranged at a forward tilted position wherein the mixing of the solutions in the first and second compartment of the module is prevented. After the end repairing process is finished, the second module is rotated back and forth to mix the magnetic beads and the end repairing reaction mix in the first compartment and the bead binding buffer in the second compartment of the third chamber. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads to the bottom of the second module at a position close to the flexible bridge B and drag the magnetic bead pellet to travel through the flexible bridge B into the first chamber of the next module.

In some NGS library preparation protocol, a PCR amplification or a reverse transcription reaction is not needed, and the first module is thus not needed. In this situation, the library preparation protocol starts from the third chamber of the second module. The DNA sample is added with the end repairing reaction mix in the first compartment of the third chamber, and the magnetic beads in a bead binding buffer are loaded in the second compartment of the third chamber. The rest of the performance is the same as described above.

The third module is used for a dA-tailing process which uses a DNA polymerase to add a single dAMP residue to the 3′ end of the blunt ended dsDNAs. The third module comprises a first chamber for receiving a washing solution, a second chamber for receiving a washing solution, and a third split chamber having a first compartment for receiving a dA-tailing reaction mix and a second compartment for receiving a bead binding buffer.

When the DNA-bound magnetic beads are transferred into the first chamber of the third module, the magnetic beads are washed in the washing solution to remove impurities. The magnetic beads are then moved into the washing solution in the second chamber for a second wash, after which the magnetic beads are moved to the dA-tailing reaction mix in the first compartment of the third chamber. The DNA molecules are incubated in the dA-tailing reaction mix at room temperature to produce dsDNAs with a single dAMP overhang at the 3′ end. All these operations are performed when the third module is arranged at a forward tilted position wherein the mixing of the solutions in the first and second compartment of the module is prevented. After the dA-tailing process is finished, the third module is rotated back and forth to mix the magnetic beads and the dA-tailing reaction mix in the first compartment and the bead binding buffer in the second compartment of the third chamber. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads to the bottom of the third module at a position close to the flexible bridge C and guide the magnetic bead pellet to travel through the flexible bridge C into the first chamber of the next module.

In some embodiment, the end repairing process and the dA-tailing process can be combined together in one module and the second module is not needed. Under this situation, when the first module is present, the first module is directly connected to the third module. The end repairing and dA-tailing reaction mix is loaded into the first compartment of the third chamber of the third module. The operation can be performed in analogy to the operations described above. When the first module is not present, the library preparation protocol starts with the third chamber of the third module. The DNA sample and the end repairing and dA-tailing reaction mix are loaded together in the first compartment of the third chamber of the third module. The magnetic beads in a bead binding reaction mix are loaded into the second compartment of the third chamber of the third module. The operation can be performed in analogy as described above.

The fourth module is used to ligate platform specific sequencing adaptors to the ends of dsDNA with 3′ overhangs. The fourth module comprises a first chamber for receiving a washing solution, a second chamber for receiving a washing solution, and a third chamber having a first compartment for receiving a ligation reaction mix and a second compartment for receiving a bead binding buffer.

When the DNA-bound magnetic beads are transferred into the first chamber of the fourth module, the magnetic beads are washed in the washing solution to remove impurities. The magnetic beads are then moved into the washing solution in the second chamber for a second wash, after which the magnetic beads are moved to the ligation reaction mix in the first compartment of the third chamber. The DNA molecules are incubated in the dA-tailing reaction mix at room temperature to produce dsDNAs with sequencing adaptors. All these operations are performed when the third module is arranged at a forward tilted position wherein the mixing of the solutions in the first and second compartment of the module is prevented. After the ligation process is finished, the fourth module is rotated back and forth to mix the magnetic beads and the ligation reaction mix in the first compartment and the bead binding buffer in the second compartment of the third chamber of the fourth module. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads to the bottom of the third module at a position close to the flexible bridge D and drag the magnetic bead pellet to travel through the flexible bridge D into the first chamber of the next module.

In some embodiment, the end repairing, the dA-tailing and DNA ligation process can be combined in one module, and the second and the third module is not needed. If the first module is present, it directly connects to the fourth module via the flexible bridge A. The first compartment of the third chamber of the fourth module is loaded with the end repairing and dA-tailing reaction mix, and the first compartment of the third chamber is connected to a MFH element that is loaded with a ligation reaction mix. After the DNA-bound magnetic beads are transferred to the first compartment of third chamber of the fourth module, the magnetic beads are incubated with the end repairing and dA-tailing reaction mix to complete the end repairing and dA-tailing process together. After that, the ligation reaction mix is transferred to the first compartment of the third chamber to perform the ligation reaction. After the ligation reaction is completed, the fourth module is rotated back and forth to mix the magnetic beads and solutions in the first compartment with the bead binding buffer in the second compartment of the third chamber of the fourth module. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. Again, the magnetic beads are pelleted and moved to the fifth module as described above.

If the first module is not present, the library preparation protocol directly starts from the third chamber of the fourth module. Under this situation, the DNA sample, the end repairing and dA-tailing reaction mix is loaded into the first compartment of the third chamber of the fourth module, and the magnetic beads in a bead binding buffer are loaded into the second compartment of the third chamber of the fourth module. The first compartment of the third chamber is connected to a MFH element that contains a ligation reaction mix. The operation in the fourth module can be performed in analogy as described above.

The fifth module is used for PCR amplification of the above dsDNA molecules with sequencing adaptors. The fifth module comprises a first chamber for receiving a washing solution, a second chamber for receiving a washing solution, and a third split chamber having a first compartment for receiving a PCR reaction mix and a second compartment for receiving a bead binding buffer, wherein a thermally controlled MFH element is connected to the first compartment of the third chamber of the fifth module.

When the DNA-bound magnetic beads are transferred into the first chamber of the fifth module, the magnetic beads are washed in the washing solution to remove impurities. The magnetic beads are then moved to the washing solution in the second chamber for a second wash, after which the magnetic beads are moved into the PCR reaction mix in the first compartment of the third chamber. The PCR reaction mix and the magnetic beads are then transferred into the connected MFH element to perform the PCR amplification. After the PCR amplification, the DNA product and the PCR reaction mix are transferred back to the first compartment. The fifth module is then rotated back and forth to mix the DNA product, the PCR reaction mix and the magnetic beads in the first compartment with the bead binding buffer in the second compartment. Optionally, an MFH element at the back of the chamber can be used to repetitively move the mixture of the reaction solution, the bead binding buffer and the magnetic beads in and out of the chamber so as to break the pellet of the magnetic beads and facilitate the DNA to be rebound to the magnetic beads. After the DNA molecules are bound to the magnetic beads, the magnet is used to pellet the DNA-bound magnetic beads to the bottom of the fifth module at a position close to the flexible bridge E and drag the magnetic bead pellet to travel through the flexible bridge E into the first chamber of the sixth module.

The sixth module is used to elute clean dsDNA molecules with adaptor oligos from the magnetic beads. The six module comprises a first chamber for receiving a washing solution, a second chamber for receiving a washing solution, and a third chamber for receiving an elution buffer.

When the DNA-bound magnetic beads are transferred into the first chamber of the sixth module, the magnetic beads are washed in the washing solution to remove impurities. The magnetic beads are then moved into the washing solution in the second chamber for a second wash, after which the magnetic beads are moved into and incubated in the elution buffer in the third chamber. The magnetic beads are then pelleted and separated from the elution buffer. The final DNA library preparation is obtained from the elution buffer.

While the present invention has been described in details and exemplified for use in the NGS library preparation, one skilled in the art will appreciate that various changes in form and detail can be made to use it in other magnetic bead-based applications (e.g. protein-related preparations) without departing from the true scope of the invention. All figures, tables, appendices, patents, patent applications and publications, referred to above, are hereby incorporated by reference.

What is claimed is:

1. A device for performing automated library preparation for next-generation sequencing, comprising:
a) a housing body;
b) at least one integrated reactor disposed inside the housing body, wherein the integrated reactor comprises more than one module wherein a module comprises at least one chamber for receiving solutions and magnetic beads, wherein adjacent chambers are separated by a chamber wall, wherein there is an opening at the apex of the chamber wall for allowing magnetic beads to pass through, wherein a chamber can be connected to at least one multi-functional helper (MFH) element;
c) a robotic hand for operating the MFH element; and
d) a magnet for moving the magnetic beads from one chamber to another chamber.

2. The device of claim 1, wherein the MFH element has fluid exchange with the connected chamber, wherein the MFH element can function as a solution loader, a mixer and/or a thermally controlled reactor.

3. The device of claim 1, wherein the MFH element is thermally coupled to a temperature controlling element.

4. The device of claim 2, wherein the MFH element uses a springy mechanism to move a solution to and from the connected chamber, and wherein the robotic hand is used to move solutions in and out of the MFH element by exerting a squeezing force.

5. The device of claim 2, wherein the MFH element uses a piston to move a solution to and from the connected chamber, wherein the piston is moved by the robotic hand.

6. The device of claim 5, wherein the piston and inner wall of the MFH element forms at least one enclosed section for receiving a solution.

7. The device of claim 1, wherein the integrated reactor can be sealed with a cover.

8. The device of claim 1, wherein the integrated reactor is disposed in a rotatable seat inside the housing body, wherein the integrated reactor can be rotated by the rotatable seat.

9. The device of claim 1, wherein the integrated reactor comprises at least two modules selected from the following modules in order:
a first module, having one chamber connected to at least an MFH element;
a second module, having a first chamber, a second chamber and a third chamber connected to at least an MFH element;
a third module, having a first chamber, a second chamber and a third chamber connected to at least an MFH element;
a fourth module, having a first chamber, a second chamber and a third chamber connected to at least an MFH element;
a fifth module, having a first chamber, a second chamber, and a third chamber connected to at least one MFH element; and
a sixth module, having a first chamber, a second chamber, and a third chamber.

10. The device of claim 9, wherein the integrated reactor comprises optionally the first module, the second module, the third module, the fourth module, optionally the fifth module, and the sixth module.

11. The device of claim 9, wherein the integrated reactor comprises optionally the first module, the third module, the fourth module, optionally the fifth module, and the sixth module.

12. The device of claim 9, wherein the integrated reactor comprises optionally the first module, the fourth module, optionally the fifth module, and the sixth module, wherein the third chamber of the fourth module is connected to a two-section MFH element or two MFH elements.

13. The device of claim 9, wherein the integrated reactor comprises the first module, the fifth module and the sixth module.

14. A method for performing automated library preparation for next-generation sequencing using a device of claim 9, comprising:
a, loading reaction solutions, magnetic beads and a nucleic acid sample into corresponding chambers or MFH elements;
b, sealing the integrated reactor and disposing it into the housing body;
c, allowing the nucleic acid sample to react in a first reaction solution to obtain a modified DNA;
d, allowing the magnetic beads to bind with the modified DNA;
e, sequentially moving the magnetic beads bound with the modified DNA from one chamber to a neighboring chamber and from one module to a neighboring module by use of the magnet, wherein, when the magnetic beads are moved to a chamber with an MFH element, the MFH element is used to add a solution to and/or mix the solution in the connected chamber; and
f, collecting the library preparation in the last chamber of the last module.

15. The method of claim 14, wherein each module, if present in the device, is loaded as the following:
the chamber of the first module is loaded with magnetic beads in a bead binding buffer, and the MFH element connected to the first module is loaded with a reaction mix and the nucleic acid sample;
the first and the second chamber of the second module are loaded with a washing solution, the third chamber of the second module is loaded with an end repairing reaction mix, and the MFH element connected to third chamber of the second module is loaded with a bead binding buffer;
the first and the second chamber of the third module are loaded a washing solution, the third chamber of the third module is loaded with a dA-tailing reaction mix, and the MFH element connected to the third chamber of the third module is loaded with a bead binding buffer;
the first and the second chamber of the fourth module are loaded with a washing solution, the third chamber of the fourth module is loaded with a ligation reaction mix, and the MFH element connected to the fourth module is loaded with a bead binding buffer;
the first and the second chamber of the fifth module are loaded with a washing solution, the third chamber of the fifth module is loaded with a PCR reaction mix, and an MFH element connected to the fifth module is loaded with a bead binding buffer; and
the first and the second chamber of the sixth module are loaded with a washing solution, and the third chamber of the sixth module is loaded with an elution buffer.

16. The method of claim 15, wherein the third chamber of the fifth module is connected to a first MFH element that is thermally controlled and left empty, and a second MFH element loaded with a bead binding buffer.

17. The method of claim 15, wherein the third chamber of the fifth module is connected to a thermally controlled two-section MFH element, wherein the first section of the MFH element is empty and the second section is loaded with a bead binding buffer.

18. The method of claim 16, wherein the operation in the fifth module comprises the steps of:
a. moving the magnetic beads into the PCR reaction mix in the third chamber of the fifth module;
b. transferring the PCR reaction mix and the magnetic beads to the first MFH element that is thermally controlled;
c. performing a PCR amplification inside the thermally controlled MFH element to obtain a PCR product;
d. transferring the magnetic beads, the PCR product and the PCR reaction mix back to the third chamber of the fifth module;
e. using the second MFH element to add the bead binding buffer and mix the magnetic beads, the PCR reaction mix and the bead binding buffer in the third chamber of the fifth module; and
f. using the magnet to pellet the magnetic beads and move them to the next module.

19. The method of claim 17, wherein the operation in the fifth module comprises the steps of:
a. moving the magnetic beads into the PCR reaction mix in the third chamber of the fifth module;
b. transferring the PCR reaction mix and the magnetic beads to the first section of the connected MFH element;
c. performing a PCR amplification inside the first section of the MFH element to obtain a PCR product;
d. transferring the PCR product, the PCR reaction mix, and the magnetic beads in the first section of the MFH element and the bead binding buffer in the second section of the MFH element into the third chamber of the fifth module;
e. using the MFH element to mix the solution and the magnetic beads in the third chamber of the fifth module; and
f. using the magnet to pellet the magnetic beads and move them to the next module.

20. The method of claim 15, wherein a device of claim 10 is used for the library preparation and the first module is not present, wherein the library preparation starts from the third chamber of the second module, and the third chamber of the second module is loaded with the nucleic acid sample and an end repairing reaction mix, and the connected MFH element is loaded with magnetic beads in a bead binding buffer.

21. The method of claim 15, wherein a device of claim 11 is used for the library preparation and the first module is present, wherein the third chamber of the third module is loaded with an end repairing and dA-tailing reaction mix.

22. The method of claim 15, wherein a device of claim 11 is used for the library preparation and the first module is not present, wherein the library preparation starts from the third chamber of the third module, wherein the third chamber of the third module is loaded with the nucleic acid sample, and an end repairing and dA-tailing reaction mix, and the connected MFH element is loaded with magnetic beads in a bead binding buffer.

23. The method of claim 15, wherein a device of claim 12 is used for the library preparation,
wherein, when the first module is present, the third chamber of the fourth module is loaded with an end repairing and dA-tailing reaction mix;
wherein, when the first module is not present, the library preparation starts from the third chamber of the fourth module, and the third chamber of the fourth module is loaded with a nucleic acid sample, and an end repairing and dA-tailing reaction mix;
wherein a ligation mix and magnetic beads in a bead binding buffer are separately loaded in the connected MFH element or MFH elements.

24. The method of claim 23, wherein the third chamber of the fourth module is connected to two MFH elements, wherein a first MFH element is loaded with the ligation mix and a second MFH element is loaded with magnetic beads in a bead binding buffer, and wherein the operation in the fourth module comprises the steps of:
a, incubating the nucleic acid sample or nucleic acid-bound magnetic beads with the end repairing and the dA-tailing reaction mix in the third chamber of the fourth module to obtain modified nucleic acids;
b, transferring the ligation reaction mix in the first MFH element to the third chamber of the fourth module to react with the modified nucleic acids;
c, using the second MFH element to add the bead binding buffer and mix the magnetic beads and the solution in the third chamber of the fourth module; and
d, using a magnet to pellet the magnetic beads and move them to the next module.

25. The method of claim 23, wherein the third chamber of the fourth module is connected to an MFH element with two sections, wherein a first section of the MFH element is loaded with the ligation mix and a second section of the MFH element is loaded with the magnetic beads in a bead binding buffer, and wherein the operation in the fourth module comprises the steps of:
a, incubating the nucleic acid sample or nucleic acid-bound magnetic beads with the end repairing and the dA-tailing reaction mix in the third chamber of the fourth module to obtain modified nucleic acids;
b, transferring the ligation reaction mix in the first section of the connected MFH element into the third chamber of the fourth module to react with the modified nucleic acids;
c, transferring the bead binding buffer in the second section of the connected MFH element into the third chamber of the fourth module;
d, using the connected MFH element to mix the solution and the magnetic beads in the third chamber of the fourth module; and
e, using a magnet to pellet the magnetic beads and move them to the next module.
